(19) United States
(12) Patent Application Publication
(10) Pub. No.: US 2006/0278248 A1
(43) Pub. Date: Dec. 14, 2006

(54) Electrophysiology catheter and system for gentle and firm wall contact

(75) Inventor: Raju R. Viswanathan, St. Louis, MO (US)

Correspondence Address: Kevin Pumm, Suite 400, 7700 Bonhomme, St. Louis, MO, 63105, US

(73) Assignee: Stereotaxis Inc.

(21) Appl. No.: 11/506,525

(22) Filed: Aug. 18, 2006

Related U.S. Application Data
(63) Continuation of application No. 11/446,522, filed on Jun. 2, 2006.
(60) Provisional application No. 60/686,786, filed on Jun. 2, 2005.

(51) Int. Cl. A61B 19/00

(52) U.S. Cl. 128/899; 600/374; 606/32

(57) ABSTRACT

A method of applying an electrode on the end of a flexible medical device to the surface of a body structure, the method including navigating the distal end of the device to the surface by orienting the distal end and advancing the device until the tip of the device contacts the surface and the portion of the device proximal to the end prolapses. Alternatively the pressure can be monitored with a pressure sensor, and used as an input in a feed back control to maintain contact pressure within a pre-determined range.

### CROSS-REFERENCE TO RELATED APPLICATIONS

This application is a continuation of U.S. patent application Ser. No. 11/446,522, Files Jun. 2, 2006, which claims the benefit of U.S. Provisional Patent Application Ser. No. 60/686,786, filed Jun. 2, 2005, the entire disclosures of which is incorporated herein by reference.

### BACKGROUND OF THE INVENTION

In intracardiac electrophysiology medical procedures, catheters have been routinely used for many years to map cardiac electrical abnormalities (arrhythmias) for diagnostic purposes, and to deliver therapy by Radio Frequency (RF) ablation of diseased tissue or abnormal electrical nodes. Usually, such catheters have been navigated within the anatomy by deflecting them with a manually operated handle, and torquing or twisting them by hand. Typically, the handle is connected to mechanical pull wires that deflect or manipulate the distal portion of the device through suitably applied tension or compression.

For certain cardiac mapping and ablation procedures the quality of the mapping and/or ablation depends upon the quality of the contact between the electrode and the cardiac tissue. It is difficult to maintain the desired contact with the moving surface of the heart during the entire cardiac cycle. Typically, relatively stiff medical devices are urged against the surface of the heart with a certain amount of force in an attempt to maintain contact during the entire cardiac cycle. This tends to locally distend the tissue during part of the cycle, and cause relatively wide variance in the contact force between the device and the tissue, potentially reducing the effectiveness of mapping and ablation. This distention may also create a local anomaly of the electrical activity that the physician is attempting to map.

### SUMMARY OF THE INVENTION

Embodiments of the devices and methods of the present invention provide improved control of the contact between a medical device and an anatomical surface, and particularly between a medical device and a moving anatomical surface.

In accordance with some embodiments of this invention, a relatively highly flexible device is used to maintain a firm but gentle contact with the anatomical surface. In one preferred embodiment a flexible medical device is navigated into contact with the anatomical surface sufficiently to remain prolapsed or buckled during the movement of the surface (e.g., during the entire cardiac cycle). If the device is radio-opaque, the prolapse can be monitored and used in feedback control of a remote navigation system to maintain satisfactory contact with the anatomical surface. The catheter may be telescoped from a relatively stiffer guide sheath.

In accordance with other embodiments of this invention, relatively stiffer medical devices are used. In one such embodiment a pressure sensor is used as feedback to maintain satisfactory contact force with the anatomical surface. The catheter may be telescoped from a relatively stiff guide sheath.

Thus, embodiments of this invention provide satisfactory and safer contact with anatomical surfaces, and in particular moving anatomical surfaces, for example for cardiac mapping, pacing, and ablation. Various embodiments provide for controlling the contact pressure in a range between predetermined minimum values and maximum values. Various embodiments also provide for telescoping the catheter from a guide sheath.

### BRIEF DESCRIPTION OF THE DRAWINGS

FIG. 1 is a schematic diagram of a first embodiment of the methods of this invention, showing the use of a prolapse to control the contact force between a medical device and an anatomical surface;

FIG. 2 is a schematic diagram of a second embodiment of the methods of this invention, showing the use of a prolapse to control the contact force between a medical device and an anatomical surface;

FIG. 3 is a schematic diagram of a third embodiment of the methods of this invention, showing the use of a contact sensor to control the contact force between a medical device and an anatomical surface;

FIG. 4 is a schematic diagram of a fourth embodiment of the methods for this invention, showing the use of a contact sensor to control contact force between a medical device and an anatomical surface;

FIG. 5A is a pre-treatment ECG chart showing an example of split potential that can be observed with the methods of this invention; and

FIG. 5B is a post-treatment ECG chart showing the successful treatment of split potential by ablation at the split potential site.

Corresponding reference numerals indicate corresponding parts throughout the several views of the drawings.

### DETAILED DESCRIPTION OF THE PREFERRED EMBODIMENT

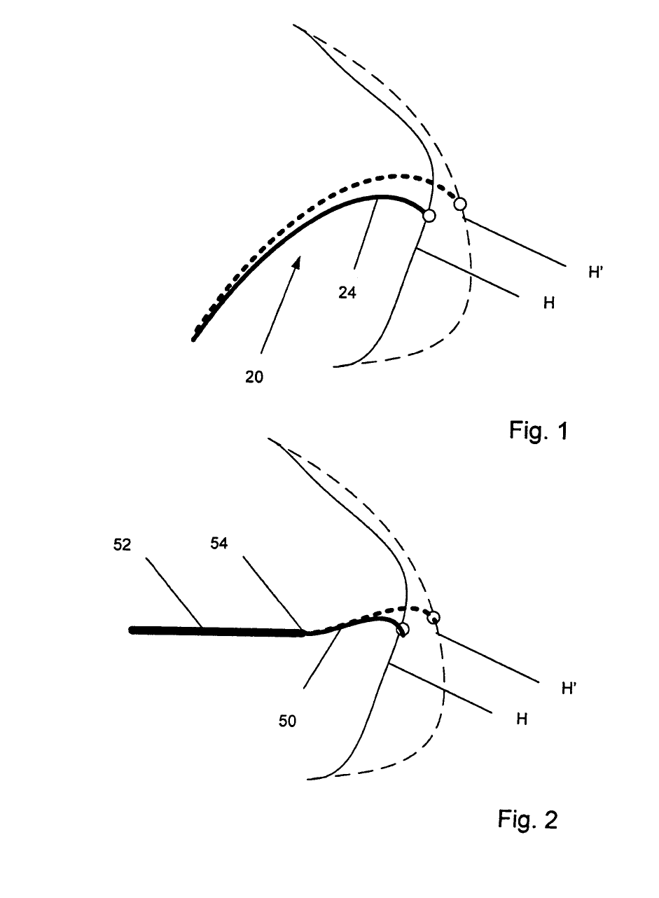

A first preferred embodiment of a catheter constructed in accordance with the principles of this invention is indicated generally as 20 in FIG. 1. The catheter 20 is preferably adapted to be navigated with a remote navigation system, such as a magnetic navigation system or a mechanical navigation system, although the catheter 20 could be manually navigated. Magnetic remote navigation is particularly advantageous because it requires only strategically placed magnetically responsive elements in the catheter, instead of mechanical control elements, and thus allows the catheters to be made more flexible. However, the invention is not limited to magnetic navigation, and includes all modes of manual and remote navigation, including mechanical, pneumatic, hydraulic, and electrostrictive navigation.

The catheter 20 preferably has at least one electrode (not shown) on its distal end. The portion 24 adjacent the distal end of relatively high flexibility. In this portion, the catheter shaft preferably has a net or effective bending modulus of 10⁻⁵ N-m² or smaller. Given the relatively small value of the bending modulus, the associated buckling force of an extended length of catheter with a 4-cm flexible length, for example, is of the order of 7 gm or smaller. When such a catheter is pushed into an anatomical surface, such as a heart wall, it cannot support forces larger than this value, minimizing the risk of wall perforation. The catheter shaft simply buckles if the user or the remote navigation system attempts to push the device into a heart wall with excessive force. In addition, avoiding excessive wall pressure is critical during RF ablation therapy, where it is essential to minimize wall pressure in sensitive areas such as the posterior wall of the left atrium, which is near the esophagus. The risk of causing complications such as esophageal fistulas is reduced when such a soft device is used.

It is possible to construct a magnetic catheter with a soft distal shaft, such as described U.S. patent application Ser. No. 10/443,113, filed May 21, 2003, entitled “Electrophysiology Catheter” Publication No. 2004-0231683 A1, dated Nov. 25, 2004, U.S. patent application Ser. No. 10/731,415, filed Dec. 9, 2003, entitled “Electrophysiology Catheter” Publication No. 2004-0147829 A1, dated Jul. 29, 2004; and U.S. patent application Ser. No. 10/865,038, filed Jun. 10, 2004, entitled “Electrophysiology Catheter” Publication No. 2004-0267106 A1, dated Dec. 30, 2004, the disclosures of which are incorporated herein by reference. A magnetic catheter can be used with a magnetic navigation system and can access a wide variety of cardiac targets. One advantage of a magnetic catheter and magnetic navigation system is the contact stability that is possible with the application of an external magnetic field. For example, in the case of the Niobe system (available from Stereotaxis, Inc., St. Louis, Mo.), the Niobe permanent magnets create the external magnetic field, and the catheter device tends to preferentially align with the magnetic field. During the cardiac cycle, the combination of the stability provided by the external magnetic field and the soft shaft of the catheter lead to consistent contact of the tip with the heart wall through the cardiac cycle. Thus, the point of contact of the catheter tip on the wall tends to remain fixed on the cardiac wall even though the wall itself is moving during the cardiac cycle. This is illustrated in FIG. 1 which shows that when the heart is contracted, the catheter 20 (shown in solid lines) contacts the wall of the heart H (shown in solid lines) at point P, and when the heart is expanded, the catheter indicated as 20′ (shown by the dashed lines) contacts the wall of the heart indicated as H′ (shown in dashed lines) still at point P. With a manual device or a stiffer device, the relative rigidity of the shaft leads to the catheter shaft retaining a relatively fixed configuration through the cardiac cycle; thus different wall points contact the catheter tip during the cardiac cycle.

By monitoring the prolapse, for example with image processing or localization, the remote navigation system can be operated to maintain a satisfactory contact force, either by determining a condition (orientation and position) in which the prolapse is maintained throughout the entire cardiac cycle, or by dynamically changing the condition (position and orientation) to maintain a prolapse as the heart wall moves. The selection of the material stiffness, and the maintenance of the prolapse also helps to control the contact force to remain between a predetermined minimum and a predetermined maximum. In this preferred embodiment, the predetermined minimum is about 3 grams, and the predetermined maximum is about 15 grams.

Alternatively, in a second embodiment, the catheter actuated by a remote navigation system can be advanced (possibly by using a joystick or other control), or magnetic field or other control variable applied, until distal catheter shaft prolapse is visible on an X-ray image or an ultrasound image. This prolapse of the catheter can be continually monitored by the user during the diagnostic process, or during the therapy delivery portion of the procedure (such as RF ablation).

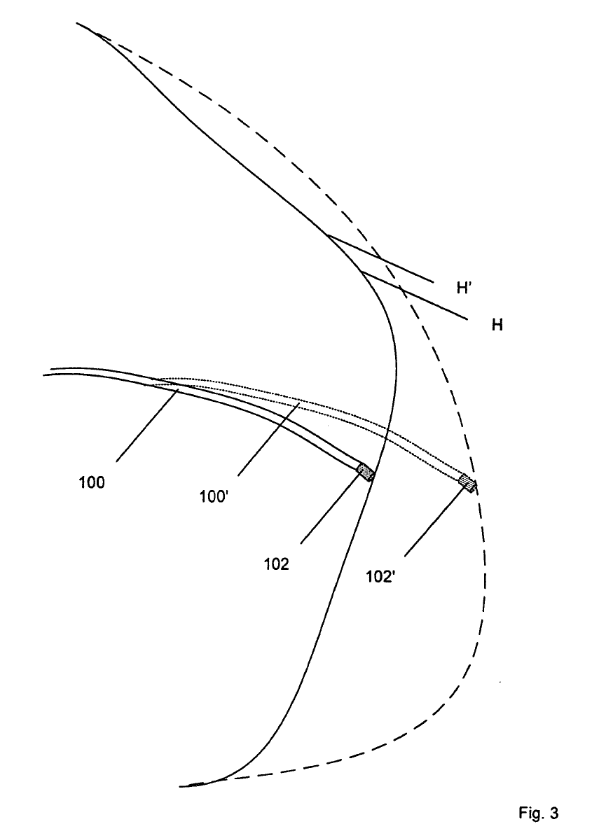

In a third embodiment shown in FIG. 2, the flexible catheter 50 is disposed inside a guide sheath 52. The guide sheath 52 is navigated to a position adjacent to and opposed to the anatomical surface of interest. This can be conveniently done with a remote navigation system, such as a magnetic navigation system or a mechanical navigation system that orients the distal end of the guide sheath. Once the distal end 54 of the guide sheath 52 is positioned, the catheter 50 is advanced until it contacts the anatomical surface and buckles. More specifically, the catheter 50 is advanced until it remains buckled during the entire cycle of movement. This is illustrated in FIG. 2 which shows that when the heart is contracted, the catheter 50 (shown in solid lines) contacts the wall of the heart H (shown in solid lines, and when the heart is expanded, the catheter indicated as 50′ (shown by the dashed lines) contacts the wall of the heart indicated as H′ (shown in dashed lines).

By monitoring the prolapse, for example with image processing or localization, the remote navigation system can be operated to maintain a satisfactory contact force, either by determining a condition (orientation and position) in which the prolapse is maintained throughout the entire cardiac cycle, or by dynamically changing the condition (position and orientation) to maintain a prolapse as the heart wall moves. The selection of the material stiffness, and the maintenance of the prolapse also helps to control the contact force to remain between a predetermined minimum and a predetermined maximum. In this preferred embodiment, the predetermined minimum is about 3 grams, and the predetermined maximum is about 15 grams.

Alternatively, in a fourth embodiment, a guide sheath actuated by the remote navigation system can be advanced (possibly by using a joystick or other control), or magnetic field or other applied control variable, until distal catheter shaft prolapse is visible on an X-ray image or an Ultrasound image. This prolapse of the catheter can be continually monitored by the user during the diagnostic process, or during the therapy delivery portion of the procedure (such as RF ablation).

Examples of a guide sheaths are disclosed in U.S. Pat. No. 6,527,782, issued Mar. 4, 2003, for “Guide for Medical Devices”, incorporated herein by reference. In one preferred embodiment the guide sheath can be actuated mechanically with pull-wire cables, as also described therein. The wires can be driven with computer-controlled servo motors or other mechanical means. The soft catheter passes through the sheath and the length of catheter that extends from the distal end of the sheath can itself be separately controlled from a proximally located advancer drive mechanism. By suitable articulation of the distal end of the sheath, the catheter tip can be navigated to various anatomical locations. Thus the articulation abilities of a mechanical remote navigation system can be combined with the navigational and contact safety advantages of a soft catheter.

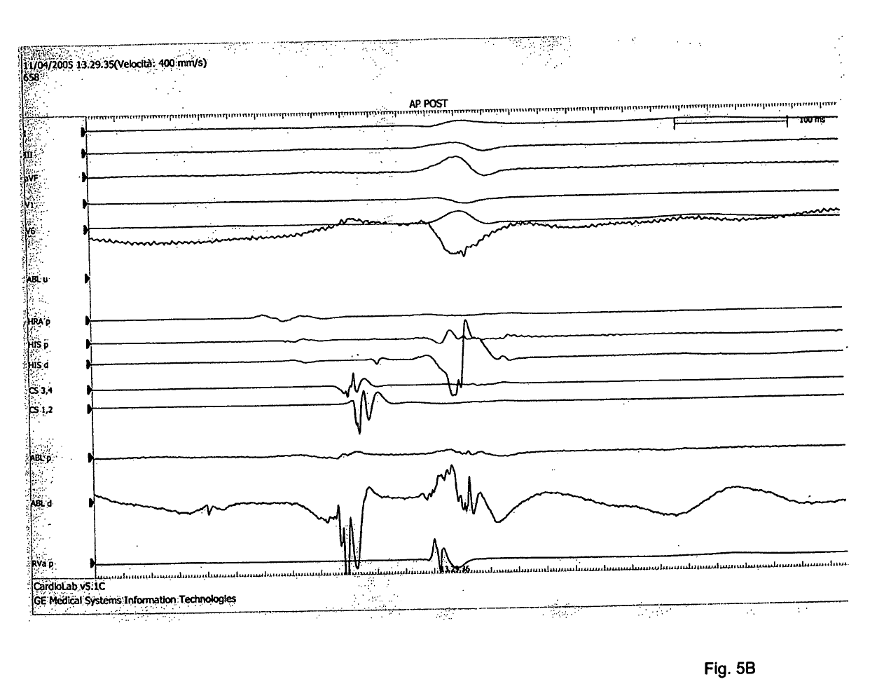

Another advantage of a soft magnetic catheter used with a magnetic navigation system is the ability to sense fine details of intracardiac ECG potentials, given the gentle but firm nature of catheter contact. An example is provided in FIG. 5A, which shows a split potential in the form of a Kent potential. Stiffer, mechanically operated devices tend to distend the cardiac wall, and further as described above the point of contact of the tip on the wall is not quite stable through the cardiac cycle. As a consequence, fine details of the local intracardiac potential tend to get smeared or lost. Magnetically driven soft catheters thus offer the possibility of more precise mapping and diagnosis in Electrophysiology procedures, along with fine, stable control of catheter contact for more precise ablation therapy delivery. FIG. 5B shows that the split potential is eliminated after ablation at the site of the split potential.

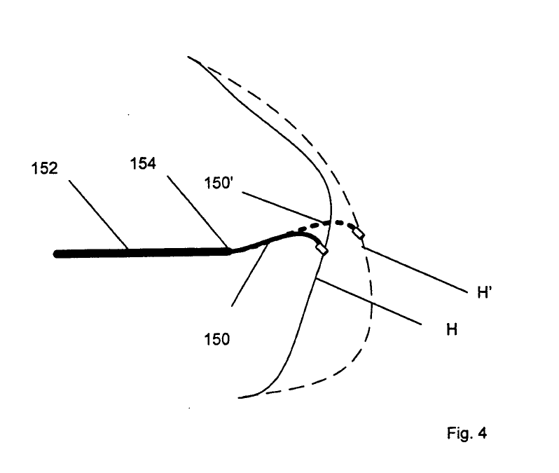

A catheter adapted for use in a fifth embodiment of this invention is indicated generally as 100 in FIG. 3. As shown in FIG. 3, the catheter 100 could have a somewhat higher bending modulus than the previously described embodiments, but it is provided with a force sensor, pressure sensor or strain gauge 102 in the catheter tip. As a safety measure, when the pressure reading from the sensor 102 exceeds a pre-determined threshold value, the remote navigation system would prevent further actuation or device advancement that might cause an increase in pressure at the tip. Alternatively or additionally, the sensed force or pressure can be displayed suitably to the user together with a warning. In this manner, gentle but firm contact could be established and maintained manually. This is illustrated in FIG. 3 which shows that when the heart is contracted, the catheter 100 (shown in solid lines) contacts the wall of the heart H (shown in solid lines) with a force measured by sensor 102, and when the heart is expanded, the catheter indicated as 100′ (shown by the dashed lines) contacts the wall of the heart indicated as H′ (shown in dashed lines) with a force measured by sensor 102.

By monitoring the force from the sensor 102, the remote navigation system can be operated to maintain a satisfactory contact force, either by determining a condition (orientation and position) in which the sensed force is maintained between predetermined minimums and maximums, throughout the entire cardiac cycle, or by dynamically changing the condition (position and orientation) to maintain the sensed force between predetermined minimums and maximums. In this preferred embodiment, the predetermined minimum is about 3 grams, and the predetermined maximum is about 15 grams.

In a sixth embodiment, the remote navigation system can actuate a sheath through which the catheter passes, and the catheter could have a somewhat higher bending modulus than given earlier. The sheath itself can be equipped with a force sensor or strain gauges that can sense changes in wall tension. Additionally or alternatively, the motors actuating the sheath can sense a change in torque as a result of contact resistance at the tip. When this force, strain or torque measurement exceeds a threshold value, further advancement of the sheath or device is prevented. The sensed force or torque can be displayed suitably to the user together with a warning.

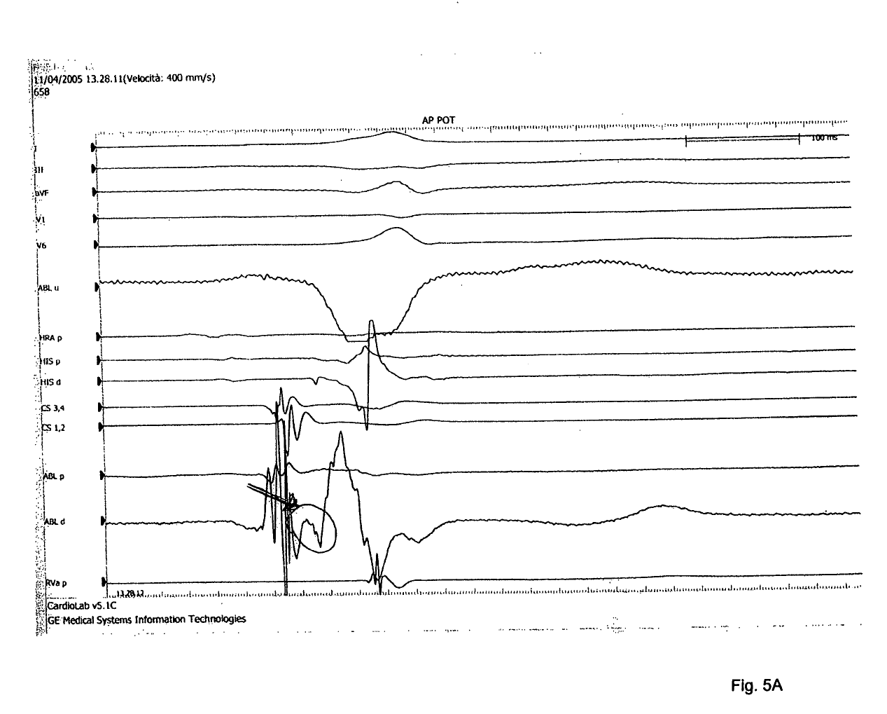

As shown in FIG. 4, a flexible catheter 150 is disposed inside a guide sheath 152. The guide sheath 152 is navigated to a position adjacent to and opposed to the anatomical surface of interest. This can be conveniently done with a remote navigation system, such as a magnetic navigation system or a mechanical navigation system that orients the distal end of the guide sheath. Once the distal end 154 of the guide sheath 152 is positioned, the catheter 150 is advanced until it contacts the anatomical surface and buckles. More specifically, the catheter 150 is advanced until it remains buckled during the entire cycle of movement. This is illustrated in FIG. 4 which shows that when the heart is contracted, the catheter 150 (shown in solid lines) contacts the wall of the heart H (shown in solid lines, and when the heart is expanded, the catheter indicated as 150′ (shown by the dashed lines) contacts the wall of the heart indicated as H′ (shown in dashed lines).

By monitoring the force from the sensor 152, the remote navigation system can be operated to maintain a satisfactory contact force, either by determining a condition (orientation and position) in which the sensed force is maintained between predetermined minimums and maximums, throughout the entire cardiac cycle, or by dynamically changing the condition (position and orientation) to maintain the sensed force between predetermined minimums and maximums. In this preferred embodiment, the predetermined minimum is about 3 grams, and the predetermined maximum is about 15 grams.

What is claimed is:

1. A method of using a remote surgical navigation system to apply an electrode on the end of a flexible medical device to the surface of a body structure, the method comprising:
navigating the distal end of the device to the surface by orienting the distal end with the remote navigation system and advancing the device until the tip of the device contacts the surface and the portion of the device proximal to the end prolapses.

2. The method according to claim 1 wherein the electrode contacts the surface with greater than about 3 grams of force and less than about 15 grams of force.

3. A method of using a remote surgical navigation system to apply an electrode on the end of a flexible medical device to the surface of a moving body structure, the method comprising:
navigating the distal end of the device to the surface by orienting the distal end with the remote navigation system and advancing the device until the tip of the device contacts the surface and the portion of the device proximal to the end remains prolapsed during the entire range of motion of the surface.

4. The method according to claim 3 wherein the electrode contacts the surface with greater than about 3 grams of force and less than about 15 grams of force.

5. A method of applying an electrode on the end of a flexible medical device to the surface of moving body structure using a remote navigation system, the method comprising navigating the distal end of the device to the surface by orienting the distal end and advancing the device using the remote navigation system, monitoring the configuration of the distal end portion of the medical device for a prolapse, and operating the remote navigations system to maintain a prolapse during the entire range of motion of the surface.

6. The method according to claim 5 wherein the remote navigation system is a magnetic navigation system that orients the distal end by applying a magnetic field to orient a magnetically responsive element on the distal end of the device.

7. An electrode catheter having an electrode on the distal end, the distal end section having a bending modulus smaller than about 10⁻⁵ N-m².

8. The electrode catheter according to claim 7 wherein the distal portion of the catheter is a radio-opaque shaft, such that any prolapse of the distal end is observable by x-ray imaging.

9. The electrode catheter according to claim 7 wherein the distal portion of the catheter is a radio-opaque shaft, such that any prolapse of the distal end is observable by x-ray imaging.

10. A method of applying an electrode on the end of a flexible medical device to the surface of moving body structure using a remote navigation system, the method comprising navigating the distal end of a guide sheath to a location facing the surface by orienting the distal end and advancing the guide sheath using the remote navigation system, deploying the flexible medical device through the guide sheath until it contacts the surface and prolapses sufficiently to maintain a prolapse during the entire range of motion of the surface.

11. The method according to claim 10 wherein the remote navigation system is a magnetic navigation system.

12. The method according to claim 10 wherein the remote navigation system uses servo motors and pull-wires to mechanically articulate the sheath

13. The method according to claim 10 wherein the remote navigation system uses electrostrictive elements to articulate the sheath.

14. An electrode catheter having an electrode at its distal tip, the portion of the catheter adjacent the distal tip having an effective bending modulus greater than 10⁻⁵ N-m² and further comprising a force sensor at the distal tip.

15. A method of applying an electrode on the end of a flexible medical device to the surface of moving body structure using a remote navigation system, the method comprising navigating the distal end of the medical device having a force sensor thereon into contact with the surface; and operating the remote navigation system to maintain the contact force between a predetermined minimum and a predetermined maximum.

16. The method according to claim 15 wherein the remote navigation system is a magnetic navigation system.

17. The method according to claim 15 wherein the remote navigation system uses servo motors and pull-wires to mechanically articulate a guide sheath through which the medical device is deployed.

18. The method according to claim 15 wherein the remote navigation system uses electrostrictive elements to articulate a guide sheath through which the medical device is deployed.

19. The method according to claim 15 wherein the force sensor includes a strain gauge.

20. A method of applying an electrode on the end of a flexible medical device to the surface of moving body structure using a remote navigation system, the method comprising navigating the distal end of a guide sheath to a location facing the surface, by orienting the distal end and advancing the device using the remote navigation system, deploying the flexible medical device having a pressure sensor thereon through the guide sheath until it contacts the surface and maintaining the contact force between the end of the medical device and the surface between a predetermined minimum and a predetermined maximum.
